(19) United States
(12) Patent Application Publication
(10) Pub. No.: US 2023/0075943 A1
(43) Pub. Date: Mar. 9, 2023

(54) IMAGING APPARATUS AND IMAGING METHOD

(71) Applicant: Shimadzu Corporation, Kyoto-shi (JP)

(72) Inventors: Kazuyuki MATSUDA, Kyoto-shi (JP); Akihiro ISHIKAWA, Kyoto-shi (JP)

(21) Appl. No.: 17/467,776

(22) Filed: Sep. 7, 2021

(51) Int. Cl. A61B 5/00

(52) U.S. Cl. CPC A61B 5/0071; A61B 5/0035; A61B 5/0077; A61B 5/7425; A61B 5/748; A61B 90/50

(57) ABSTRACT

The imaging apparatus includes: a first light source; a second light source; a reference scale disposed in close proximity to a subject; a first imaging unit for capturing a visible image; a second imaging unit for capturing a fluorescence image; an image display unit for displaying a visible image and a fluorescence image; an operation unit which is a user interface for inputting a desired measurement line on the fluorescence image; and a distance calculation unit for calculating a length of the measurement line based on a length of the reference scale, and the image display unit displays a length of the measurement line calculated by the distance calculation unit.

### RELATED APPLICATION

This application is related to JP 2019-038528 filed in Japan on Mar. 4, 2019, the entire contents of which are herein incorporated by reference in their entirety.

### TECHNICAL FIELD

The present invention relates to an imaging apparatus and an imaging method.

### BACKGROUND ART

A method called near-infrared fluorescence imaging has been used for angiography in surgery. In this near-infrared fluorescence imaging, indocyanine green (ICG), which is a fluorescent dye, is administered to an affected part by injection with an injector or the like. When this indocyanine green is irradiated with near-infrared light having a wavelength of about 600 nm to 850 nm (nm: nanometers) as excitation light, the indocyanine green emits near-infrared fluorescence having a wavelength of about 750 nm to 900 nm. The fluorescence is imaged by an imaging element capable of detecting near-infrared rays, and the image is displayed on a display unit, such as, e.g., a liquid crystal display panel. According to the near-infrared fluorescence imaging, it becomes possible to observe blood vessels, lymphatic vessels, and the like existing at a depth of about 20 mm from the body surface.

For example, Patent Document 1 discloses a method of obtaining a graph showing a temporal change in fluorescence intensity and generating a color image (color map) based on various indices, such as, e.g., a slope, a time to a peak, and an area of the graph. Then, in a case where the method described in Patent Document 1 is used, the color image becomes an image in which the color is continuously changed, for example, according to the blood circulation state of the subject. With reference to this color image, the doctor can determine where the blood circulation has deteriorated, i.e., where the surgical procedure should be performed on the blood vessel.

### PRIOR ART DOCUMENT

### Patent Document

Patent Document 1: Japanese Patent No. 5,918,532

### SUMMARY OF THE INVENTION

### Problems to be Solved by the Invention

For example, when it is desired to grasp the thickness of a blood vessel and the size of an organ, it is not possible to accurately perform the same only by the color image obtained using the method described in Patent Document 1.

The present invention has been made to solve the above-described problems. An object of the present invention is to provide an imaging apparatus and an imaging method capable of accurately grasping size of a measurement target object whose size is required to be grasped.

### Means for Solving the Problem

A first aspect of the present invention relates to an imaging apparatus comprising:

a first light source configured to emit white light to a subject;

a second light source configured to emit excitation light to the subject, the excitation light being for exciting a fluorescent dye injected in the subject;

a reference scale configured to be arranged in proximity to the subject;

a first imaging unit configured to capture a visible image by imaging the subject irradiated with the white light;

a second imaging unit configured to capture a fluorescence image by imaging fluorescence generated from the fluorescent dye;

an image display unit configured to display the visible image and the fluorescence image;

a user interface configured to input a desired measurement line on the fluorescence image displayed on the image display unit; and

a distance calculation unit configured to calculate a length of the measurement line based on a length of the reference scale, the reference scale having been imaged by the first imaging unit or the second imaging unit,

wherein the image display unit displays the length of the measurement line calculated by the distance calculation unit.

A second aspect of the present invention relates to an imaging apparatus comprising:

a first light source configured to emit white light to a subject;

a second light source configured to emit excitation light to the subject, the excitation light being for exciting a fluorescent dye injected in the subject;

a reference scale configured to be arranged in proximity to the subject;

a first imaging unit configured to capture a visible image by imaging the subject irradiated with the white light;

a second imaging unit configured to capture a fluorescence image by imaging fluorescence generated from the fluorescent dye;

an image display unit configured to display the visible image and the fluorescence image;

a user interface configured to input a reference line along the reference scale on the visible image displayed on the image display unit and input a desired measurement line on the fluorescence image displayed on the image display unit; and

a distance calculation unit configured to calculate a length of the measurement line based on a length of the reference scale,

wherein the image display unit displays the length of the measurement line calculated by the distance calculation unit.

A third aspect of the present invention relates to an imaging method comprising the steps of:

emitting white light to a subject;

emitting excitation light to the subject, the excitation light being for exciting a fluorescent dye injected in the subject;

capturing a visible image by imaging the subject irradiated with the white light;

capturing a fluorescence image by imaging fluorescence generated from the fluorescent dye;

displaying the visible image and the fluorescence image on an image display unit;

inputting a desired measurement line on the fluorescence image displayed on the image display unit; and

calculating a length of the measurement line based on a length of the reference scale, the reference scale having been imaged in a step of capturing the visible image or a step of capturing the fluorescence image; and

displaying the length of the measurement line calculated by the distance calculation unit on the image display unit.

A fourth aspect of the present invention relates to an imaging method comprising the steps of:

emitting white light to a subject;

emitting excitation light to the subject, the excitation light being for exciting a fluorescent dye injected in the subject;

capturing a visible image by imaging the subject irradiated with the white light;

capturing a fluorescence image by imaging fluorescence generated from the fluorescent dye;

displaying the visible image and the fluorescence image on an image display unit;

inputting a reference line along the reference scale on the visible image displayed on the image display unit and inputting a desired measurement line on the fluorescence image displayed on the image display unit;

calculating a length of the measurement line based on the length of the reference scale, the reference scale having been imaged in a step of capturing the visible image or a step of capturing the fluorescence image; and

displaying the length of the measurement line calculated by the distance calculation unit on the image display unit.

### Effects of the Invention

According to the present invention, in a case where there is a measurement target object whose size is desired to be grasped, the size of the measurement target object can be grasped accurately.

### BRIEF DESCRIPTION OF THE DRAWINGS

FIG. 1 is a perspective view showing an embodiment of an imaging apparatus of the present invention.

FIG. 2 is a side view of the imaging apparatus shown in FIG. 1.

FIG. 3 is a plan view of the imaging apparatus shown in FIG. 1.

FIG. 4 is a block diagram showing a main control system of the imaging apparatus shown in FIG. 1.

FIG. 5 is a diagram showing the steps in order performed by the imaging apparatus shown in FIG. 1.

FIG. 6 is an example of an image displayed on an image display unit provided in the imaging apparatus shown in FIG. 1.

### EMBODIMENTS FOR CARRYING OUT THE INVENTION

Hereinafter, an imaging apparatus and an imaging method of the present invention will be described in detail based on a preferred embodiment shown in the accompanying drawings.

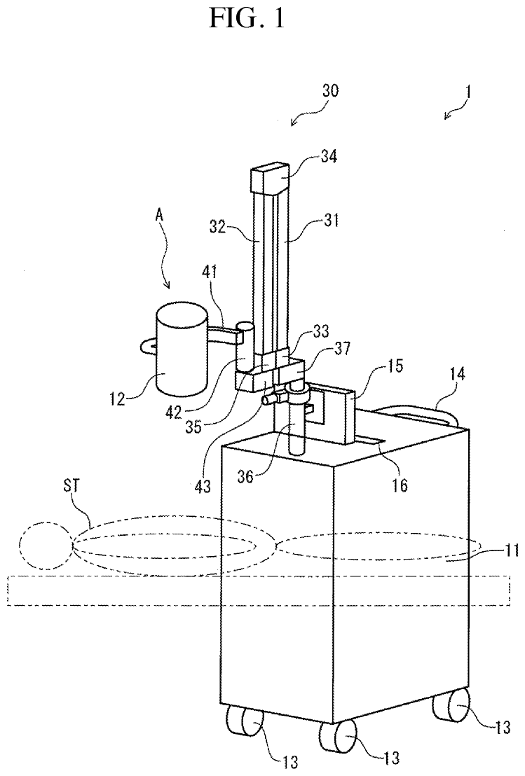

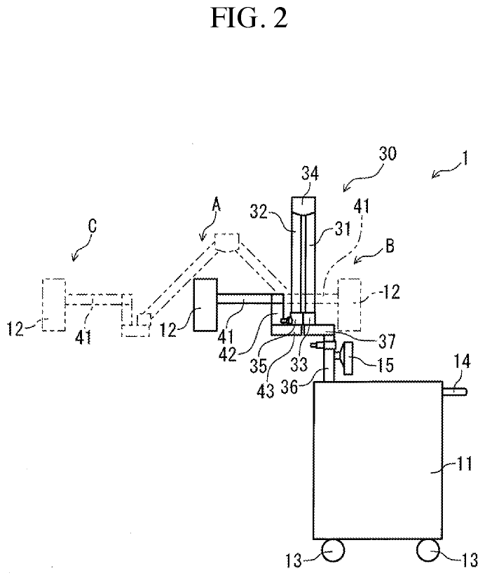

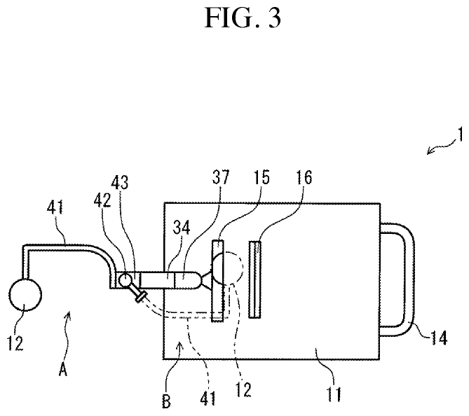

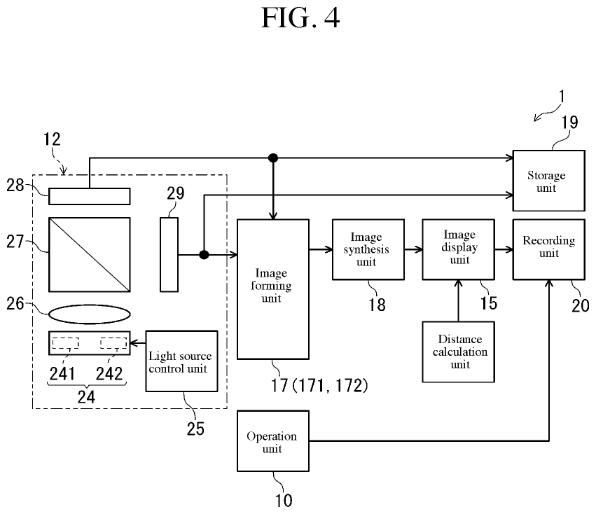

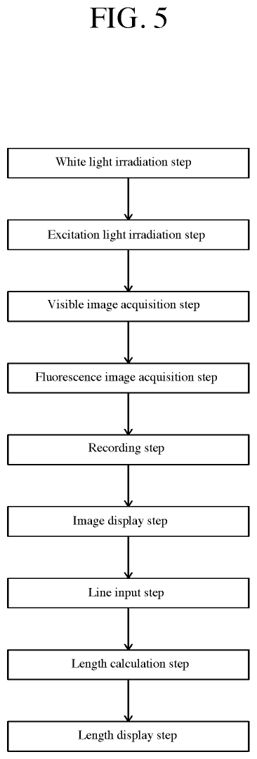

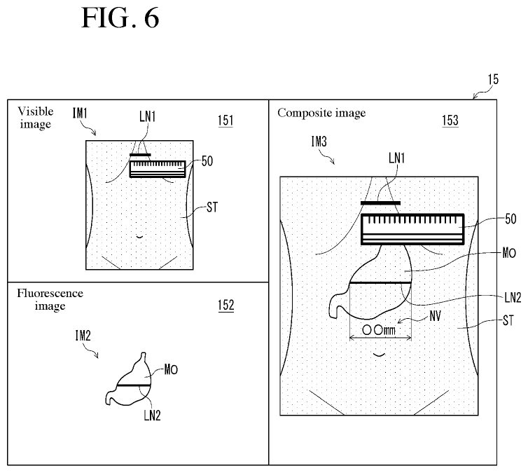

FIG. 1 is a perspective view showing an embodiment of an imaging apparatus of the present invention. FIG. 2 is a side view of the imaging apparatus shown in FIG. 1. FIG. 3 is a plan view of the imaging apparatus shown in FIG. 1. FIG. 4 is a block diagram showing a main control system of the imaging apparatus shown in FIG. 1. FIG. 5 is a diagram showing the steps in order performed by the imaging apparatus shown in FIG. 1. FIG. 6 is an example of an image displayed on an image display unit included in the imaging apparatus shown in FIG. 1. Note that in the following description, for convenience of description, the upper side in FIGS. 1, 2, and 6 is referred to as “upper,” and the lower side is referred to as “lower”.

The imaging apparatus 1 shown in FIG. 1 is an apparatus configured to emit excitation light to indocyanine green (ICG) as a fluorescent dye injected in a body of a subject ST and image the fluorescence emitted from the indocyanine green. By using the imaging apparatus 1, it is possible to accurately grasp the blood circulation state of the subject ST when, for example, performing surgery on the subject ST.

The imaging apparatus 1 is provided with a carriage 11, an arm mechanism 30, an lighting and imaging unit 12, and an image display unit 15. The carriage 11 is provided with four wheels 13. The arm mechanism 30 is disposed near the front (in the left direction in FIGS. 2 and 3) of the carriage 11 in the traveling direction on the upper surface of the carriage 11. The lighting and imaging unit 12 is disposed via a sub-arm 41 to the arm mechanism 30. The image display unit 15 is a monitor. At the rear portion of the carriage 11 in the traveling direction, a handle 14 for moving the carriage 11 is attached. Further, on the upper surface of the carriage 11, the recess 16 for mounting a remote control for remotely operating the imaging apparatus 1 is formed.

As shown in FIG. 5, the imaging apparatus 1 can execute an imaging method including the following steps. The steps include a white light irradiation step, an excitation light irradiation step, a visible image acquisition step, a fluorescence image acquisition step, a recording step, an image display step, a line input step, a length calculation step, and a length display step. The imaging method performs the above steps in that order.

The above-described arm mechanism 30 is provided on the front side of the carriage 11 in the traveling direction. The arm mechanism 30 is provided with a first arm member 31 connected, via a hinge portion 33, to the support portion 37 disposed on the support 36 erected on the front side of the carriage 11 in the traveling direction. The first arm member 31 is swingable with respect to the carriage 11 via the support 36 and the support portion 37 by the action of the hinge portion 33. Note that the above-described image display unit 15 is attached to the support 36.

At the upper end of the first arm member 31, a second arm member 32 is connected by a hinge portion 34. The second arm member 32 is swingable with respect to the first arm member 31 by the action of the hinge portion 34. Therefore, the first arm member 31 and the second arm member 32 can take an imaging posture and a standby posture. The imaging posture is a posture in which, as shown by the imaginary line denoted by the symbol C in FIG. 2, the first arm member 31 and the second arm member 32 are opened at a predetermined angle centering the hinge portion 34, which is a connecting portion between the first arm member 31 and the second arm member 32. The standby posture is a posture in which as indicated by the solid line denoted by the symbol A in FIGS. 1 to 3, the first arm member 31 and the second arm member 32 are close to each other.

At the bottom of the second arm member 32, a support portion 43 is connected by a hinge portion 35. The support portion 43 is swingable with respect to the second arm member 32 by the action of the hinge portion 35. A rotation shaft 42 is supported by the support portion 43. A sub-arm 41 supporting the lighting and imaging unit 12 is rotatable about the rotation shaft 42 disposed at the distal end of the second arm member 32. Therefore, the lighting and imaging unit 12 moves between the position on the rear side of the carriage 11 in the traveling direction with respect to the arm mechanism 30 and the position on the front side of the carriage 11 in the traveling direction with respect to the arm mechanism 30 by the rotation of the sub-arm 41. The position on the front side of the carriage 11 in the traveling direction is a position for taking an imaging posture or a standby posture as indicated by the solid line denoted by the symbol A in FIGS. 1 to 3 or an imaginary line denoted by the symbol C in FIG. 2. Further, the position on the rear side of the carriage 11 in the traveling direction is a posture for moving the carriage 11, as shown by the imaginary line with a sign B in FIGS. 2 and 3.

As shown in FIG. 4, the lighting and imaging unit 12 is provided with a light source unit 24, a light source control unit 25, a zoom lens 26, a prism 27, a white light sensor 28, and an excitation light sensor 29. When the imaging apparatus 1 is used, it is preferable that the lighting and imaging unit 12 be separated from the affected part of the subject ST by about several tens of centimeters.

The light source unit 24 is provided with a first light source 241 that is a white light source and a second light source 242 that is a light source for excitation.

The first light source 241 emits white light. With this, a white light irradiation step of emitting white light toward the subject ST can be performed. This white light is reflected from the subject ST as reflected light.

The second light source 242 irradiates excitation light to excite the fluorescent dye. Thus, an excitation light irradiation step of irradiating excitation light for exciting the fluorescent dye can be performed toward the subject ST to which the fluorescent dye has been injected and administered. In a case where the fluorescent dye is indocyanine green, for example, near-infrared rays having a wavelength of 810 nm is preferably used as the excitation light for exciting the indocyanine green. From the indocyanine green irradiated with the 810 nm near-infrared ray, a near-infrared ray having a peak of about 845 nm is emitted as fluorescence.

The light source control unit 25 has a function of controlling the lighting of the first light source 241. With this function, it is possible to cause the first light source 241 to start emission of white light and stop emission of white light. Further, the light source control unit 25 has a function of controlling the lighting of the second light source 242. With this function, it is possible to case the second light source 242 to start emission of excitation light and stop emission of excitation light. Note that the light source control unit 25 is configured by, for example, a CPU or the like.

White light, which is the reflected light reflected by the subject ST, and fluorescence, which is the near-infrared ray generated from the indocyanine green administered in the subject ST, are incident on the zoom lens 26. With this zoom lens 26, the white light is focused on the white light sensor 28, and the fluorescence is focused on the excitation light sensor 29.

The light from the zoom lens 26, that is, the white light and the fluorescence are collectively incident on the prism 27. The collectively incident white light and fluorescence are separated by the prism 27 in such a manner that the white light is directed to the white light sensor 28, and the fluorescence is directed to the excitation light sensor 29.

The white light sensor 28 detects a part of the white light separated by the prism 27. Excitation light sensor 29 detects a part of the near-infrared ray (excitation light) separated by the prism 27.

Further, as shown in FIG. 4, the imaging apparatus 1 is provided with an image forming unit 17, an image synthesis unit 18, a storage unit 19, a recording unit 20, and an operation unit 10. These units are arranged on the carriage 11.

The white light detected by the white light sensor 28 and the near-infrared fluorescence detected by the excitation light sensor 29 are input to the image forming unit 17. Then, the image forming unit 17 forms the white light detected by the white light sensor 28 as a 24-bit (=3×8) visible image IM1 composed of three colors of RGB (red, green, and blue). The image forming unit 17 forms the fluorescence detected by the excitation light sensor 29 as a fluorescence image IM2 of 8 bits. As described above, in this embodiment, the image forming unit 17 functions as a first imaging unit 171 which is a visible image imaging unit, and functions as a second imaging unit 172 which is a fluorescence image imaging unit. The first imaging unit 171 captures a visible image IM1 by imaging the subject ST irradiated with the white light. The second imaging unit 172 captures a fluorescence image IM2 by imaging the fluorescence generated from the fluorescent dye.

In the first imaging unit 171, a visible image acquisition step for capturing the visible image IM is performed by imaging the subject ST irradiated with the white light. In the second imaging unit 172, a fluorescence image acquisition step of capturing the fluorescence image IM2 is performed by imaging the fluorescence generated from the fluorescent dye.

The image synthesis unit 18 synthesizes the visible image IM1 by the white light formed by the image forming unit 17 and the fluorescence image IM2 by the fluorescence to form (generate) a composite image IM3.

As shown in FIG. 6, the visible image IM1, the fluorescence image IM2, and the composite image IM3 are collectively displayed on the image display unit 15. As a result, it is possible to perform an image display step of displaying the visible image IM1, the fluorescence image IM2, and the composite image IM3 on the image display unit 15. In this embodiment, the image display unit 15 is divided into a first display section 151 for displaying the visible image IM1, a second display section 152 for displaying the fluorescence image IM2, and a third display section 153 for displaying the composite image IM3. Then, by observing the composite image IM3 in the third display section 153, the doctor can accurately grasp the blood circulation state of the subject ST, and can accurately determine the portion where the blood vessel is to be separated, that is, the separation line, based on the blood circulation state.

The storage unit 19 is configured to store the white light (signal) detected by the white light sensor 28 and the fluorescence (signal) detected by the excitation light sensor 29.

The recording unit 20 is configured to record the image displayed on the image display unit 15. As a result, it is possible to perform a recording step of recording the visible image IM1, the fluorescence image IM2, and the composite image IM3. Then, the doctor can review any of the recorded visible image IM1, the recorded fluorescence image IM2, and the recorded composite image IM3 on the image display unit 15 as required, thereby performing an accurate operation.

The operation unit 10 is a user interface for performing the operation of the imaging apparatus 1. For example, the operation unit 10 is configured to operate the start of the irradiation of the light from the light source unit 24, the stop of the irradiation, the adjustment of the brightness and sensitivity, the display method of the image displayed on the image display unit 15, etc.

As described above, by using the imaging apparatus 1, the blood circulation state of the subject ST can be grasped when performing surgery on the subject ST. Further, in the surgical operation, for example, the thickness of a blood vessel, the size of an organ, and the like may be sometimes desired to be grasped. Therefore, the imaging apparatus 1 is configured to be able to grasp the size of the measurement target object MO when there is a measurement target object MO whose size is required to be grasped. Hereinafter, the configuration and operation of the imaging apparatus will be described. Note that, although the measurement target object MO is exemplified by a “stomach” in this embodiment, the measurement target object MO is not limited thereto.

As shown in FIG. 6, the imaging apparatus 1 is provided with a reference scale 50 to be arranged in close proximity to the subject ST. The reference scale 50 is not particularly limited, and for example, when it is desired to measure the width of the stomach which is the measurement target object MO, it is preferable to use a straight-line ruler, but depending on the measurement target object MO, other measurement instruments, such as, e.g., a fractional instrument, can be used.

The reference scale 50 is placed, for example, on the chest of the subject ST. Then, in the visible image acquisition step, the subject ST and the reference scale 50 on the subject ST are imaged in a state of being irradiated with the white light. As a result, it is possible to capture the visible image IM1 in which the subject ST and the reference scale 50 are reflected. The visible image IM is displayed on the first display section 151 of the image display unit 15.

The operation unit 10, which is a user interface, includes a mouse (not shown). By using the mouse, the user can enter a reference line LN1 along the reference scale 50 on the visible image IM1 displayed on the first display section 151 of the image display unit 15. The length of the reference line LN1 may be a length from the position 0 to any position of the scale of the reference scale 50. The length of the reference line LN1 is preferably shorter than the total length of the reference scale 50, i.e., preferably shorter than the maximum measurement length. The length of the reference line LN1 at this time can be input as a numerical value.

In addition, in the same manner as in the input of the reference line LN1, a desired measurement line LN2 can be input on the fluorescence image IM2 displayed on the second display section 152 of the image display unit 15 by using a mouse. In other words, in this embodiment, it is possible to input the measurement line LN2 at a position corresponding to the width of the stomach.

As described above, in the imaging apparatus 1, the reference line LN1 along the reference scale 50 can be input on the visible image IM1 displayed on the image display unit 15. Further, a desired measurement line LN2 can be input on the fluorescence image IM2 displayed on the image display unit 15. Thus, the line input step is performed.

In this embodiment, the reference scale 50 is imaged by the first imaging unit 171 and appears in the visible image IM1, but the invention is not limited thereto. For example, in a case where a fluorescent material is applied to the scale of the reference scale 50, the reference scale 50 may be imaged by the second imaging unit 172 to be reflected in the fluorescence image IM2.

As shown in FIG. 4, the imaging apparatus 1 is provided with a distance calculation unit 60. The distance calculation unit 60, calculates (operates) the length of the measurement line LN2 based on the length of the reference line LN1. Note that the distance calculation unit 60 is configured by, for example, a CPU or the like.

This calculation method is not particularly limited, and examples thereof include the following methods.

First, the number of pixels within the length of the reference line LN1 (hereinafter referred to as “first pixel number”) and the number of pixels within the length of the measurement line LN2 (hereinafter referred to as “second pixel number”) are detected. Next, how many times the number of the second pixels becomes the number of the first pixels is calculated. The result of this calculation is then multiplied by the length of the actual reference lines LN1. With this, the length of the actual measurement lines LN2 is acquired.

For example, in a case where the first pixel number is 100 pixels and the second pixel number is 150 pixels, the second pixel number is 1.5 times the first pixel number. Then, if the length of the actual reference line LN1 is 50 mm, the length of the actual measurement line LN2 is calculated as 75 mm by the calculation of 50 mm×1.5 times.

Thus, in the imaging apparatus 1, based on the length of the reference line LN1, it is possible to calculate the length of the measurement line LN2. Thus, the length calculation step is performed.

Note that the distance calculation unit 60 can calculate the length of the measurement line LN2 based on the length of the reference scale 50, without using the length of the reference line LN1. The calculation method in this case is also the same as the calculation method described above.

The length of the measurement lines LN2 is displayed on the third display section 153 of the image display unit 15 as a numerical value NV indicating the actual length. Thus, the length display step of displaying the length of the measurement line LN2 calculated by the distance calculation unit 60 on the third display section 153 of the image display unit 15 is performed.

By visually recognizing the numerical value NV displayed on the third display section 153, the doctor can accurately grasp the width of the stomach, which is the measurement target object MO. As a result, the operation can be performed safely and accurately.

As described above, when there is a measurement target object MO whose size is desired to be grasped, the size of the measurement target object MO can be grasped accurately by using the imaging apparatus 1.

Although the imaging apparatus and the imaging method of the present invention have been described above with reference to the illustrated embodiment, the present invention is not limited thereto. In addition, each part constituting the imaging apparatus can be replaced with any part which can exhibit the same function. An optional component may also be added.

Further, in the above-described embodiment, an example is shown in which indocyanine green is used as a material containing a fluorescent dye, and near-infrared ray of about 600 nm to 850 nm is emitted to this indocyanine green as excitation light to thereby cause the indocyanine green to emit the fluorescence in a near-infrared region having a peak of about 810 nm. However, in the present invention, light other than near-infrared rays may be used.

Further, depending on the case of the subject, instead of using the indocyanine green as a fluorescent dye, for example, a 5-aminolevulinic acid (5-ALA/5-Aminolevulinic Acid) may be used.

### [Aspects]

It will be understood by those skilled in the art that the plurality of exemplary embodiments described above is illustrative of the following aspects.

### (Item 1)

An imaging apparatus (1) according to one embodiment includes:

a first light source (241) configured to emit white light to a subject;

a second light source (242) configured to emit excitation light to the subject, the excitation light being for exciting a fluorescent dye injected in the subject;

a reference scale (50) configured to be arranged in proximity to the subject;

a first imaging unit (171) configured to capture a visible image by imaging the subject irradiated with the white light;

a second imaging unit (172) configured to capture a fluorescence image by imaging fluorescence generated from the fluorescent dye;

an image display unit (15) configured to display the visible image and the fluorescence image;

a user interface (10) configured to input a desired measurement line on the fluorescence image displayed on the image display unit; and

a distance calculation unit (60) configured to calculate a length of the measurement line based on a length of the reference scale, the reference scale having been imaged by the first imaging unit or the second imaging unit,

wherein the image display unit displays the length of the measurement line calculated by the distance calculation unit.

According to the imaging apparatus described in the above-described Item 1, when there is a measurement target object whose size is desired to be grasped, the size of the measurement target object can be grasped accurately.

### (Item 2)

The imaging apparatus (1) as recited in the above-described Item 1, further comprising:

a recording unit (20) configured to record the visible image and the fluorescence image,

wherein the image display unit displays the visible image and the fluorescence image recorded in the recording unit.

According to the imaging apparatus described in the above-described Item 2, the size of the measurement target object in the recorded visible image and the recorded fluorescence image can be accurately grasped.

### (Item 3)

An imaging apparatus (1) according to one aspect of the present invention, comprising:

a first light source (241) configured to emit white light to a subject;

a second light source (242) configured to emit excitation light to the subject, the excitation light being for exciting a fluorescent dye injected in the subject;

a reference scale (50) configured to be arranged in proximity to the subject;

a first imaging unit (171) configured to capture a visible image by imaging the subject irradiated with the white light;

a second imaging unit (172) configured to capture a fluorescence image by imaging fluorescence generated from the fluorescent dye;

an image display unit (15) configured to display the visible image and the fluorescence image;

a user interface (10) configured to input a reference line along the reference scale on the visible image displayed on the image display unit and input a desired measurement line on the fluorescence image displayed on the image display unit; and

a distance calculation unit (60) configured to calculate a length of the measurement line based on the length of the reference scale,

wherein the image display unit displays the length of the measurement line calculated by the distance calculation unit.

According to the imaging apparatus described in the above-described Item 3, the size of the measurement target object can be accurately grasped based on the length of the reference line.

### (Item 4)

The imaging apparatus as recited in the above-described Item 3, further comprising:

a recording unit (20) configured to record the visible image and the fluorescence image,

wherein the image display unit displays the visible image and the fluorescence image recorded in the recording unit.

According to the imaging apparatus described in the above-described Item 4, the size of the measurement target object in the recorded visible image and the recorded fluorescence image can be accurately grasped.

### (Item 5)

An imaging method according to one embodiment of the present invention, comprising the steps of:

emitting white light to a subject;

emitting excitation light to the subject, the excitation light being for exciting a fluorescent dye injected in the subject;

capturing a visible image by imaging the subject irradiated with the white light;

capturing a fluorescence image by imaging fluorescence generated from the fluorescent dye;

displaying the visible image and the fluorescence image on an image display;

inputting a desired measurement line on the fluorescence image displayed on the image display unit; and

calculating a length of the measurement line based on the length of the reference scale, the reference scale having been imaged in a step of capturing the visible image or a step of capturing the fluorescence image; and

displaying the length of the measurement line calculated by the distance calculation unit on the image display unit.

According to the imaging method described in the above-described Item 5, when there is a measurement target object whose size is desired to be grasped, the size of the measurement target object can be grasped accurately.

### (Item 6)

The imaging method as recited in the above-described Item 5, further comprising the step of:

recording the visible image and the fluorescence image,

wherein in a step of displaying the visible image and the fluorescence image on the image display unit, the visible image and the fluorescence image recorded in the recording unit are displayed.

According to the imaging apparatus described in the above-described Item 6, the size of the measurement target object in the recorded visible image and the recorded fluorescence image can be accurately grasped.

### (Item 7)

An imaging method according to one embodiment of the present invention, comprising the steps of:

emitting white light to a subject;

emitting excitation light to the subject, the excitation light being for exciting a fluorescent dye injected in the subject;

capturing a visible image by imaging the subject irradiated with the white light;

capturing a fluorescence image by imaging fluorescence generated from the fluorescent dye;

displaying the visible image and the fluorescence image on an image display;

inputting a reference line along the reference scale on the visible image displayed on the image display unit and inputting a desired measurement line on the fluorescence image displayed on the image display unit;

calculating a length of the measurement line based on the length of the reference scale, the reference scale having been imaged in a step of capturing the visible image or a step of capturing the fluorescence image; and

displaying the length of the measurement line calculated by the distance calculation unit on the image display unit.

According to the imaging apparatus described in the above-described Item 7, the size of the measurement target object can be accurately grasped based on the length of the reference line.

### (Item 8)

The imaging method as recited in the above-described Item 7, further comprising the step of:

recording the visible and fluorescence images,

wherein in a step of displaying the visible image and the fluorescence image on the image display unit, the visible image and the fluorescence image recorded in the recording unit are displayed.

According to the imaging apparatus described in the above-described Item 8, the size of the measurement target object in the recorded visible image and the recorded fluorescence image can be accurately grasped.

### DESCRIPTION OF SYMBOLS

1: Imaging apparatus
10: Operation Unit
11: Carriage
12: Lighting and imaging unit
13: Wheel
14: Handle
15: Image display unit
   151: First display section
   152: Second display section
   153: Third display section
16: Recess
17: Image forming unit
   171: First imaging unit
   172: Second imaging section
18: Image synthesis unit
19: Storage area
20: Recording unit
24: Light source unit
   241: First light source
   242: Second light source
25: Light source control unit
26: Zoom lens
27: Prism
28: White light sensor
29: Excitation light sensor
30: Arm mechanism
31: First arm member
32: Second arm member
33: Hinge portion
34: Hinge portion
35: Hinge portion
36: Support
37: Support portion
41: Sub-arm
42: Rotation shaft
43: Support portion
50: Reference scale
60: Distance calculation unit
IM1: Visible image
IM2: Fluorescence image
IM3: Composite image
LN1: Reference line
LN2: Measurement line
MO: Measurement target object
NV: Numerical value
ST: Subject

1. An imaging apparatus comprising:
a first light source configured to emit white light to a subject;
a second light source configured to emit excitation light to the subject, the excitation light being for exciting a fluorescent dye injected in the subject;
a reference scale configured to be arranged in proximity to the subject;
a first imaging unit configured to capture a visible image by imaging the subject irradiated with the white light;
a second imaging unit configured to capture a fluorescence image by imaging fluorescence generated from the fluorescent dye;
an image display unit configured to display the visible image and the fluorescence image;
a user interface configured to input a desired measurement line on the fluorescence image displayed on the image display unit; and
a distance calculation unit configured to calculate a length of the measurement line based on a length of the reference scale, the reference scale having been imaged by the first imaging unit or the second imaging unit,
wherein the image display unit displays the length of the measurement line calculated by the distance calculation unit.

2. The imaging apparatus as recited in claim 1, further comprising:
a recording unit configured to record the visible image and the fluorescence image,
wherein the image display unit displays the visible image and the fluorescence image recorded in the recording unit.

3. An imaging apparatus comprising:
a first light source configured to emit white light to a subject;
a second light source configured to emit excitation light to the subject, the excitation light being for exciting a fluorescent dye injected in the subject;
a reference scale configured to be arranged in proximity to the subject;
a first imaging unit configured to capture a visible image by imaging the subject irradiated with the white light;
a second imaging unit configured to capture a fluorescence image by imaging fluorescence generated from the fluorescent dye;
an image display unit configured to display the visible image and the fluorescence image;
a user interface configured to input a reference line along the reference scale on the visible image displayed on the image display unit and input a desired measurement line on the fluorescence image displayed on the image display unit; and
a distance calculation unit configured to calculate a length of the measurement line based on a length of the reference scale,
wherein the image display unit displays the length of the measurement line calculated by the distance calculation unit.

4. The imaging apparatus as recited in claim 3, further comprising:
a recording unit configured to record the visible image and the fluorescence image,
wherein the image display unit displays the visible image and the fluorescence image recorded in the recording unit.

5. An imaging method comprising the steps of:
emitting white light to a subject;
emitting excitation light to the subject, the excitation light being for exciting a fluorescent dye injected in the subject;
capturing a visible image by imaging the subject irradiated with the white light;
capturing a fluorescence image by imaging fluorescence generated from the fluorescent dye;
displaying the visible image and the fluorescence image on an image display unit;
inputting a desired measurement line on the fluorescence image displayed on the image display unit; and
calculating a length of the measurement line based on a length of the reference scale, the reference scale having been imaged in a step of capturing the visible image or a step of capturing the fluorescence image; and
displaying the length of the measurement line calculated by the distance calculation unit on the image display unit.

6. The imaging method as recited in claim 5, further comprising the step of:
recording the visible image and the fluorescence image,
wherein in a step of displaying the visible image and the fluorescence image on the image display unit, the visible image and the fluorescence image recorded in the recording unit are displayed.

7. An imaging method comprising the steps of:
emitting white light to a subject;
emitting excitation light to the subject, the excitation light being for exciting a fluorescent dye injected in the subject;
capturing a visible image by imaging the subject irradiated with the white light;
capturing a fluorescence image by imaging fluorescence generated from the fluorescent dye;
displaying the visible image and the fluorescence image on an image display unit;
inputting a reference line along the reference scale on the visible image displayed on the image display unit and inputting a desired measurement line on the fluorescence image displayed on the image display unit;
calculating a length of the measurement line based on the length of the reference scale, the reference scale having been imaged in a step of capturing the visible image or a step of capturing the fluorescence image; and
displaying the length of the measurement line calculated by the distance calculation unit on the image display unit.

8. The imaging method as recited in claim 7, further comprising the step of:
recording the visible and fluorescence images,
wherein in a step of displaying the visible image and the fluorescence image on the image display unit, the visible image and the fluorescence image recorded in the recording unit are displayed.
